(19) United States
(12) Patent Application Publication
(10) Pub. No.: US 2025/0381008 A1
(43) Pub. Date: Dec. 18, 2025

(54) MARKER AND INTRODUCER FOR MAGNETIC LOCALIZATION

(71) Applicant: Stryker Corporation, Portage, MI (US)

(72) Inventors: Michael GIANNINI, Toronto (CA); Ananth RAVI, Toronto (CA); John DILLON, Mississauga (CA)

(73) Assignee: Stryker Corporation, Portage, MI (US)

(21) Appl. No.: 19/236,743

(22) Filed: Jun. 12, 2025

Related U.S. Application Data
(60) Provisional application No. 63/659,327, filed on Jun. 13, 2024.

(51) Int. Cl. A61B 90/00

(52) U.S. Cl. CPC A61B 90/39; A61B 2090/3954; A61B 2090/3987

(57) ABSTRACT

A deployment system for a magnetic marker includes a cannula having a lumen, an open distal end, and a at least one lateral aperture; and a magnetic marker having a body and at least one resilient prong extending from the body, the at least one resilient prong configured to have an expanded state and a compressed state, wherein the magnetic marker is configured to be stored in the expanded state within the lumen of the cannula such that at least a portion of the at least one resilient prong is within a corresponding lateral aperture of the at least one lateral aperture of the cannula, wherein the lumen has a diameter configured to compress the at least one resilient prong of the magnetic marker into the compressed state during deployment of the magnetic marker.

### CROSS-REFERENCE TO RELATED APPLICATIONS

This application claims the benefit of U.S. Provisional Application No. 63/659,327, filed Jun. 13, 2024, the entire contents of which is incorporated herein by reference.

### FIELD

The present disclosure relates to markers for localization, and more specifically, to magnetic markers used for surgical localization.

### BACKGROUND

Magnetic markers have been deployed commercially and designed to identify a soft tissue lesion for subsequent surgical excision. Wire-free magnetic markers allow the radiological implantation of the marker to occur on a different day than the surgical removal of the marker. The separation of the implantation and surgery procedures results in an improved experience for patients and providers alike. However, for certain surgical sites such as in the colon where tissues regenerate and the internal lumen continually gets replaced or in lung tissue with increased vascularity, the increased time between the implantation and the surgical removal of the marker raises concerns about marker migration. If the marker migrates or is dislodged due to normal tissue regeneration or vascularity, it will lead to inaccurate identification of the intended target lesion and compromise the therapeutic goals of the treatment.

### SUMMARY

According to an aspect, a deployment system for a magnetic marker includes a magnetic marker having a body and at least one resilient prong extending from the body. The at least one resilient prong is configured to have an expanded state and a compressed state. The magnetic marker is configured to be stored in the expanded state within the lumen of a cannula such that at least a portion of the at least one resilient prong is within a corresponding lateral aperture of the cannula. The lumen has a diameter configured to compress the at least one resilient prong of the magnetic marker into the compressed state during deployment of the magnetic marker. Upon deployment of the magnetic marker in tissue of a subject, the at least one resilient prong expands into a deployed state in which the at least one resilient prong engages with the tissue and helps prevent the magnetic marker from migrating or dislodging from its deployed position.

According to an aspect, a deployment system for a magnetic marker includes a cannula having a lumen, an open distal end, and a at least one lateral aperture; and a magnetic marker having a body and at least one resilient prong extending from the body, the at least one resilient prong configured to have an expanded state and a compressed state, wherein the magnetic marker is configured to be stored in the expanded state within the lumen of the cannula such that at least a portion of the at least one resilient prong is within a corresponding lateral aperture of the at least one lateral aperture of the cannula, wherein the lumen has a diameter configured to compress the at least one resilient prong of the magnetic marker into the compressed state during deployment of the magnetic marker.

The magnetic marker may have two resilient prongs, and the cannula may have two lateral apertures. The body of the magnetic marker may include a permanent magnet. The permanent magnet may have a polymer overmold.

The at least one resilient prong of the magnetic marker may have an extension. The extension may include a projection that is configured to contact an inner surface of the lumen when the at least one resilient prong is in the compressed state within the lumen.

The expanded state may be the same as a deployed state of the marker. The at least one lateral aperture may include a distal edge that is oriented at an angle that is non-perpendicular with a longitudinal direction of the cannula. The magnetic marker may include a drug.

The deployment system may include a plurality of magnetic markers stored within the lumen. The deployment may include an obturator for insertion into a proximal end of the lumen and configured to push the magnetic marker during deployment.

According to an aspect, a marker includes a body; and at least one resilient prong extending from the body, the at least one resilient prong configured to have an expanded state and a compressed state, wherein the marker is configured to be stored in the expanded state within a lumen of a cannula such that at least a portion of the at least one resilient prong is within a corresponding lateral aperture of a at least one lateral aperture of the cannula.

The marker may have two resilient prongs. The marker may include a permanent magnet. The permanent magnet may have a polymer overmold. The at least one resilient prong may have an extension. The extension may include a projection that is configured to contact an inner surface of the lumen when the at least one resilient prong is in the compressed state within the lumen. The marker may include a drug.

According to an aspect, a method for deployment system of a marker includes positioning a cannula at a location of a subject, the cannula having stored therein a marker, the at least one marker having a body and at least one resilient prong extending from the body, the at least one resilient prong configured to have an expanded state and a compressed state, wherein the magnetic marker is stored in the expanded state within a lumen of the cannula such that at least a portion of the at least one resilient prong is within a corresponding lateral aperture of at least one lateral aperture of the cannula; and moving an obturator distally to eject the marker from the cannula and deploy the marker at the location of the subject.

The magnetic marker may have two resilient prongs, and the cannula may have two lateral apertures. The body of the magnetic marker may include a permanent magnet. The permanent magnet may have a polymer overmold. The at least one resilient prong of the magnetic marker may have an extension. The extension may include a projection that contacts an inner surface of the lumen when the at least one resilient prong is in the compressed state within the lumen. The method of claim 19, wherein the expanded state is the same as a deployed state of the marker. The at least one lateral aperture may include a distal edge that is oriented at an angle that is non-perpendicular with a longitudinal direction of the cannula. The magnetic marker may include a drug. The cannula may store a plurality of magnetic markers, and the method may include repositioning the cannula to deploy the magnetic markers at different locations of the subject.

According to an aspect, a deployment system for a marker includes a cannula having a lumen, an open distal end, and a narrowed region; and a marker configured to engage with the narrowed region such that engagement between the marker and the narrowed region retains the marker within the lumen.

According to an aspect, a deployment system for a marker includes a cannula having a lumen and an open distal end; and a marker comprising at least one magnet configured to magnetically engage with at least a portion the lumen such that engagement between the at least one magnet and the lumen retains the marker within the lumen.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE DRAWINGS

For a fuller understanding of the nature and objects of the disclosure, reference should be made to the following detailed description taken in conjunction with the accompanying drawings, in which:

FIG. 1A is a perspective view of a marker according to an embodiment of the present disclosure;

FIG. 1B is a side elevation view of the marker of FIG. 1A.

FIG. 1C is a front view of the marker of FIGS. 1A and 1B.

FIG. 1D is a cross-sectional view of the marker of FIGS. 1A-1C—the cross-section is taken along A-A of FIG. 1B.

FIG. 1E is a is a cross-sectional view of the marker of FIGS. 1A-1D—the cross-section is taken along B-B of FIG. 1C.

FIG. 1F is a bottom view of the marker of FIGS. 1A-1E.

FIG. 1G is a is a top view of the marker of FIGS. 1A-1F.

FIG. 2A is a view of a cannula according to an embodiment of the present disclosure.

FIG. 2B is another view of the cannula of FIG. 2A, wherein the view is rotated 90° about a longitudinal axis.

FIG. 3A is a partial view of a cannula having a marker disposed within.

FIG. 3B is a cross-sectional view of the cannula and marker of FIG. 3A.

FIG. 3C is another partial view of the cannula and marker of FIGS. 3A and 3B, wherein the view is rotated 90° about a longitudinal axis.

FIG. 3D is a cross-sectional view of the cannula and marker of FIG. 3C.

FIG. 4A depicts a marker in the compressed state.

FIG. 4B depicts a marker in the expanded state.

FIG. 5 depicts multiple views of a cannula according to another embodiment of the present disclosure, each at a different rotation about a longitudinal axis.

FIG. 6 is a detail view of the cannula tip of FIG. 5.

FIG. 7 is a perspective view of an exemplary cannula with a marker positioned within.

FIG. 8 is a cross section of an exemplary cannula and an exemplary marker positioned within the cannula.

FIG. 9A illustrates a marker that includes a drug-loaded substance. FIG. 9B illustrates an exemplary marker that includes both a magnet for magnetic localization and a drug-loaded substance. FIG. 9C illustrates an exemplary marker that includes a drug coating that coats a portion of an outer surface of the marker.

FIG. 9D illustrates an example of cannula that includes a drug-loaded composition deposited within a distal end of the cannula.

FIG. 10A illustrates an example in which a cannula includes a narrowed region configured to retain a marker within the cannula.

FIG. 10B illustrates an example in which a marker having resilient prongs is retained within a cannula via a narrowed region.

FIG. 10C illustrates an example in which a marker includes one or more magnets that are configured to magnetically attach to the inner surface of a cannula to retain the marker within the cannula.

FIG. 11A illustrates an example of multiple markers loaded into a cannula.

FIG. 11B illustrates the cannula of FIG. 10A loaded with multiple markers.

FIG. 11C illustrates an exemplary cannula that includes multiple narrowed regions for retaining multiple markers within the cannula.

### DETAILED DESCRIPTION

In the following description of the various examples, reference is made to the accompanying drawings in which are shown, by way of illustration, specific examples that can be practiced. The description is presented to enable one of ordinary skill in the art to make and use the invention and is provided in the context of a patent application and its requirements. Various modifications to the described examples will be readily apparent to those persons skilled in the art, and the generic principles herein may be applied to other examples. Thus, the present invention is not intended to be limited to the examples shown but is to be accorded the widest scope consistent with the principles and features described herein.

Described herein are marker deployment systems for deploying markers in tissue of a subject, such as for magnetic localization. A deployment system may include a cannula and marker configured to retain the marker in the cannula. A marker may include a body and at least one resilient prong extending from the body. The at least one resilient prong may be configured to have an expanded state and a compressed state. The marker may be configured to be stored in the expanded state within the lumen of a cannula such that at least a portion of the at least one resilient prong is within a corresponding lateral aperture of the cannula. The lumen may have a diameter configured to compress the at least one resilient prong of the marker into the compressed state during deployment of the marker in tissue of a subject. Upon deployment of the marker in tissue of a subject, the at least one resilient prong may expand into a deployed state in which the at least one resilient prong engages with the tissue and helps prevent the marker from migrating or dislodging from its deployed position. Thus, the resilient prong may serve multiple functions-retention of the marker within the lumen and retention of the marker within tissue.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units, but do not preclude the presence or addition of one or more other features, integers, steps, operations, elements, components, units, and/or groups thereof.

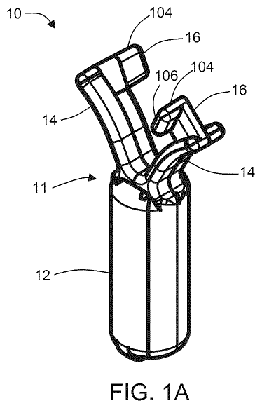

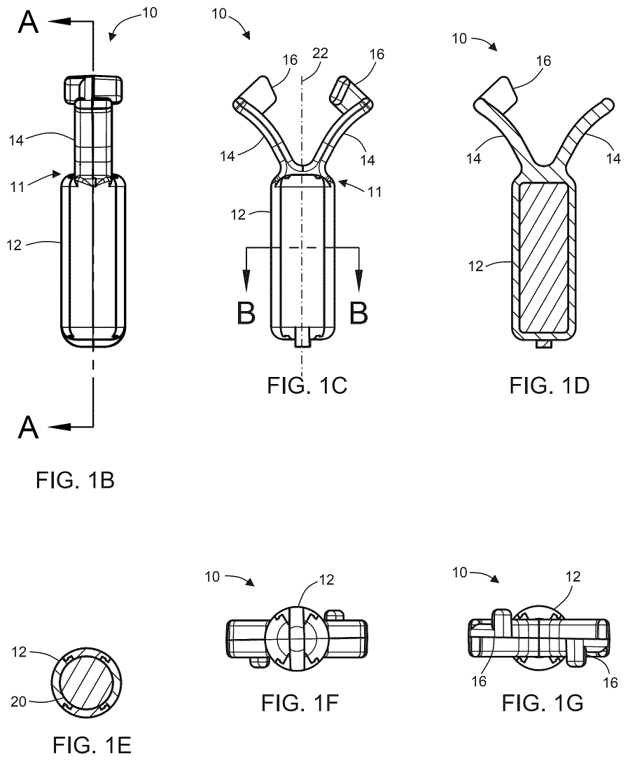

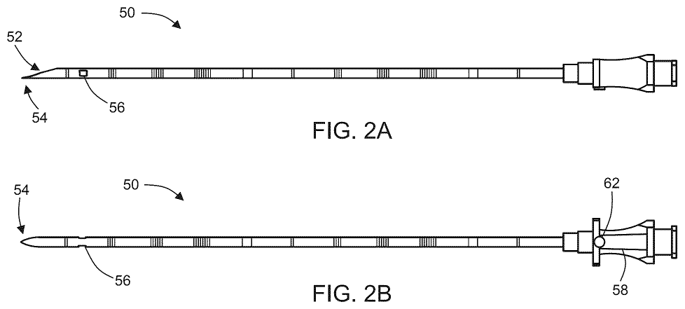

With reference to FIGS. 1A-1G, 2A, and 2B, the present disclosure may be embodied as a deployment system for a magnetic marker 10. The system includes a cannula 50 (i.e., an introducer, a needle, or an introducer needle) having a lumen 52 and an open distal end 54. The cannula 50 includes at least one lateral aperture 56. In some embodiments, the cannula 50 includes two lateral apertures 56. Other embodiments may include more than two lateral apertures.

The system also includes a marker 10, which may be a magnetic marker. The magnetic marker 10 has a body 12, which may include a magnet 20. For example, the body may be made up of a permanent magnet with a polymer overmold (e.g., polypropolene). The body may be elongate having a longitudinal axis 22 extending between a first end and a second end. At least one resilient prong 14 extends from an end 11 of the body 12. The at least one prong may be resilient in that it may be bent when a lateral force is applied—for example, bent towards the longitudinal axis—and recover to its original shape when the force is removed. In this way, the marker 10 may have an expanded state, wherein the marker has a first overall diameter (across the longitudinal axis) and a compressed state, wherein the marker has a second overall diameter which is less than the first overall diameter.

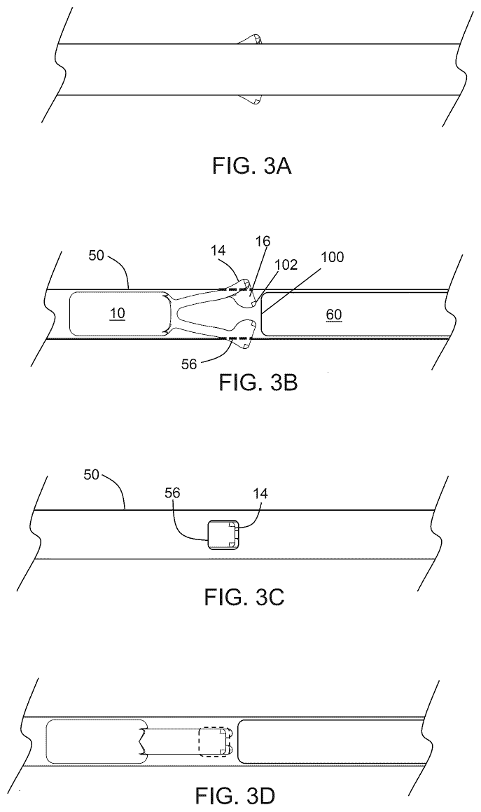

As can be seen in FIGS. 3A-3D, the marker 10 is configured to be stored in the expanded state within the lumen 52 of the cannula 50. In this stored configuration, at least a portion of the resilient prong 14 is within (i.e., passes through) a corresponding lateral aperture 56 of the cannula 50. The embodiment depicted in FIGS. 3A-3D has a marker with two resilient prongs, each prong located within a corresponding lateral aperture of the cannula. In this way, the marker may be stored in its expanded state and avoid a potential deformation and/or loss of resilience during storage due to, for example, plastic creep.

The system may include an obturator 60 operable to deploy the marker 10. For example, an obturator may be configured for insertion into a proximal end of the lumen and to move (translate) through the lumen to push the marker out of the cannula from the open distal end. When pushed from the stored configuration, the cannula wall (inner wall forming the lumen) will cause the resilient prong(s) to the compressed state because the resilient prongs can no longer pass through the lateral apertures. In such an embodiment, the compressed state of the marker has a diameter which is the same as a diameter of the lumen. Once ejected from the cannula, the marker can take on a deployed state having a third diameter. The third diameter (of the deployed state) may be the same as or different from the first diameter (of the expanded state). The third diameter may be greater than the second diameter (of the compressed state). As such, the marker may be deployed using a cannula which has a narrow diameter (relative to a deployed state of the marker).

In some embodiments, the obturator is located within the lumen and adjacent to the stored marker. The obturator may have a slidable handle 62 accessible via a slot 58 of the cannula. In this way an operator may use the slidable handle to move the obturator and deploy the marker.

Upon deployment of the marker 10 in tissue of a subject, the at least one resilient prong 14 expands into a deployed state in which the at least one resilient prong 14 engages with the tissue and helps prevent the marker 10 from migrating or dislodging from its deployed position. The marker 10 can be used for magnetic localization using a suitable magnetic localization system that is configured to detect the magnet 20.

The marker may be doped with a contrast agent for improved visualization during imaging (e.g., mammogram). The marker may include a fluorescence agent that enables visualization of the marker using a fluorescence imaging system. The fluorescence agent may be included in a coating on an outer surface of the marker, such as an outer surface of body 12.

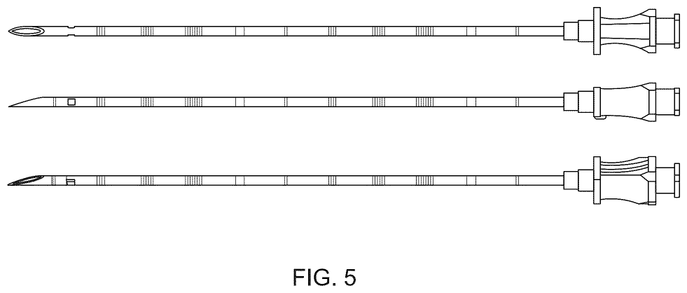

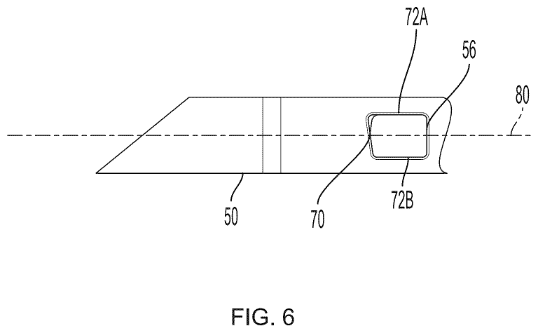

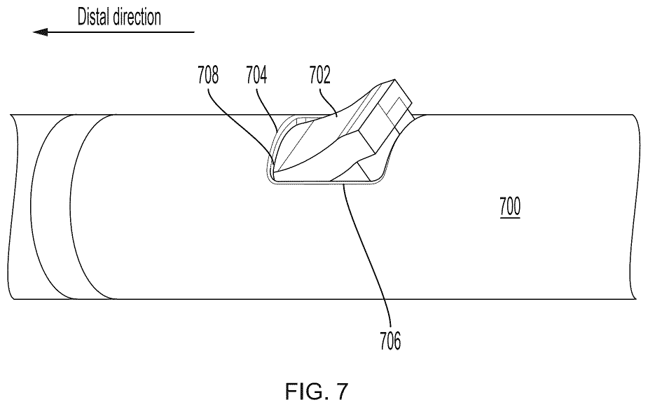

FIG. 5 depicts multiple views of a cannula according to non-limiting embodiments, each at a different rotation about a longitudinal axis. FIG. 6 illustrates that the aperture 56 may have an angled distal edge 70 that is oriented at an angle that is non-perpendicular with a longitudinal direction of the cannula 50 such that the distal edge 70 is oriented non-perpendicularly with respect to the longitudinally extending side edges 72A and 72B of the aperture 56. In the illustrated embodiment, the distal edge 70 is angled at 10 degrees from a plane extending perpendicularly to the longitudinal axis 80 of the cannula 50. However, this is merely exemplary and it should be understood that a shallower or steeper angle may be used. The angling of the distal edge 70 may facilitate deployment of the marker 10 by reducing the friction force between the distal edge 70 and the resilient prong 14 extending through the aperture 56. The angling of the distal edge 70 results in only a proximal portion of the distal edge 70 contacting the resilient prong 14 extending through the aperture 56. In other words, with the proximal portion of the distal edge 70 in contact with the resilient prong 14, a distal portion of the distal edge 70 will be spaced apart from the resilient prong 14. Thus, the resilient prong 14 makes less contact with the distal edge 70 than it otherwise would if the distal edge 70 were perpendicular to the side edges 72A and 72B. This reduction in contact between the distal edge 70 and the resilient prong 14 reduces the friction between the components, making it easier for the resilient prong 14 to slide past the distal edge 70 upon deployment of the marker 10. FIG. 7 is a perspective view of an exemplary cannula 700 with a marker positioned within, illustrating the contact between a resilient prong 702 of the marker contacting a proximal portion 708 of an angled distal edge 704 of an aperture 706 of the cannula 700. The resilient prong 702 contacts only the proximal portion 708 of the distal edge 704. As such, the contact between the distal edge 704 and the resilient prong 702 is limited to the proximal portion 708 of the distal edge 704.

Referring to FIGS. 1A-G, 3B, and 4A-B, at least one resilient prong 14 may include an inwardly extending extension 16 that is configured to engage with the obturator 60 to facilitate deployment of the marker 10. The extension 16 extends inwardly (i.e., toward a longitudinal axis of the marker 10) from an inwardly facing surface of the resilient prong 14. The extension 16 may ensure that the obturator 60 does not inadvertently insert between the resilient prongs 14 when moving distally to deploy the marker 10. With reference to FIG. 3B, the extension 16 may be configured such that the distal end 100 of the obturator 60 contacts a rounded proximal edge 102 of the extension 16. Upon distal movement of the obturator 60 that applies a distal force to the marker 10, the rounded proximal edge 102 of the extension 16 slides on the distal end 100 of the obturator 60 as the resilient prongs 14 are compressed inwardly. Thus, the extensions 16 both ensure that the obturator 60 does not inadvertently insert in between the resilient prongs 14 (which would tend to spread them apart) and facilitate compression of the resilient prongs 14 upon deployment of the marker 10 by the obturator 60.

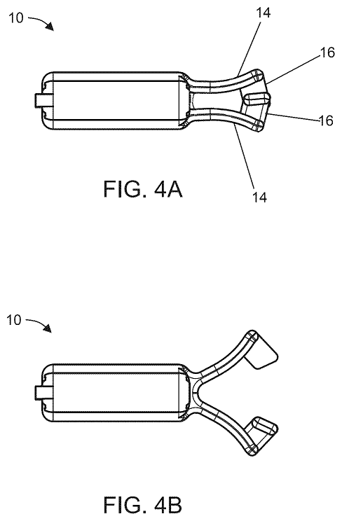

The extensions 16 may be offset from one another in the lateral direction such that the extensions 16 can be positioned alongside one another when the resilient prongs 14 are in compressed positions. An example of the extensions 16 extending alongside one another is illustrated in FIG. 4A.

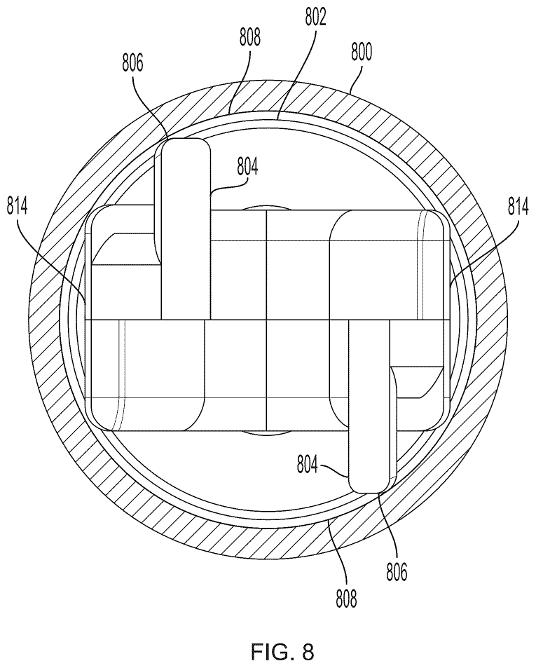

Referring to FIG. 1A, the extension 16 may include a projection 104 that extends in a lateral direction from an inner-most edge of the extension 16. With the projection 104, the extension 16 may form an L-shape. The projections 104 of opposite extensions 16 may extend in opposite lateral directions. The projection 104 may provide structural rigidity to the extension 16 and may help center the resilient prongs 14 via engagement with the inner surface of the cannula 50. For example, the lateral edge 106 of the projection 104 may abut the inner surface of the cannula 50 when the marker 10 is positioned within the cannula 50. FIG. 8 is a cross section of an exemplary cannula 800 and an exemplary marker 802 (which may be marker 10) positioned within the cannula 800. FIG. 8 illustrates contact between lateral sides 806 of projections 804 contacting the inner surface 808 of the cannula 800. This contact between the lateral sides 806 and the inner surface 808 of the cannula 800 ensures that the resilient prongs 814 do not flex outwardly in a direction transverse to their designed flexing direction (i.e., the direction of flexing from the compressed state to the expanded state), which may be advantageous in preventing unwanted flexing or bending of the resilient prongs 814 when the obturator is applying a force to the resilient prongs 814.

The permanent magnet may be made from a neodymium. The permanent magnet may be coated with a bio-compatible coating, such as, for example, a polymer overmold. In some embodiments, the permanent magnet includes one or more stacked magnetic segments. Each magnetic segment may be coated with a bio-compatible coating.

Markers may be used in various applications, such as for marking tumors in breast tissue, in the colon, or in the lungs. As noted above in reference to FIGS. 1A-G, marker 10 can include a magnet 20 that can be used to locate the marker 10 within a subject using a suitable magnetic localization system. In some examples, the marker does not include a magnet. For example, a marker configured for use as a biopsy clip may not include a magnet. Markers for different applications may have different sizes. For example, a marker for marking tumors in breast tissue may be sized for loading into a 14-gauge cannula and a marker for use in the lungs (e.g., for use in video assisted thoracic surgery (VATS)) may be sized for loading into an 18-gauge cannula).

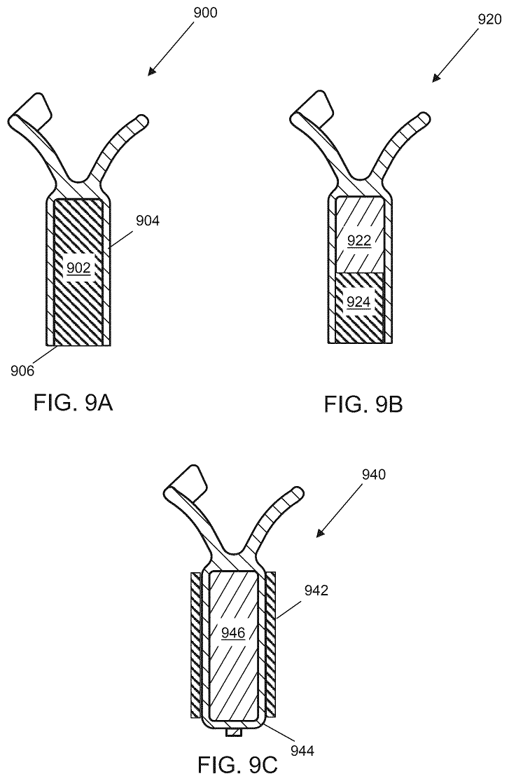

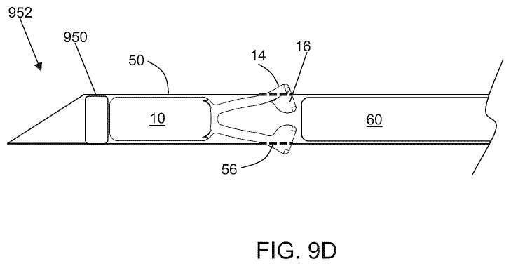

A marker may be configured for drug delivery. For example, a marker may have a drug coating and/or may carry a drug within. FIG. 9A illustrates a marker 900 that is similar to marker 10 but includes a drug-loaded composition 902 within the body 904. An end 906 of the body 904 may be open such that the drug-loaded composition 902 can move out of the marker 900 and into surrounding tissue. FIG. 9B illustrates an exemplary marker 920 that includes both a magnet 922 for magnetic localization and a drug-loaded composition 924. Thus, marker 920 can be used for delivering a drug to a location of a subject (e.g., a tumor) and for marking the location. FIG. 9C illustrates an exemplary marker 940 that includes a drug coating 942 coating a portion of an outer surface of the body 944 of the marker 940. Marker 940 may include a magnet 946 for magnetic localization. The drug-loaded composition 902, drug-loaded composition 924, and/or drug coating 942 can be, for example, a polymer loaded with a drug that is configured to absorb into tissue or a hydrogel that includes a drug that is configured to absorb into tissue. Any of the markers described herein can include a drug-loaded composition. Any suitable drugs may be used. In some examples, the drug is an anti-tumor drug, such as a chemotherapeutic drug. The drug may be an immunotherapeutic drug or a radionuclide. In some examples, a drug-loaded composition is located at the distal end of a cannula (e.g., cannula 50 of FIG. 2A), distally of a marker, and the deployment of the marker deposits the drug within the tissue of the patent at the same location as the marker.

Optionally, a drug-loaded composition can be deposited within a distal end of a cannula, distal of a marker, such that when the marker is ejected from the cannula, the drug-loaded composition is also ejected. FIG. 9D illustrates an example of cannula 50 that includes a drug-loaded composition 950 deposited within a distal end 952 of the cannula 50. When the obturator 60 pushes the marker 10 out of the cannula 50, the drug-loaded composition 950 will be pushed out as well, such that the drug-loaded composition 950 is deposited at the same location of the subject at which the marker 10 is deployed.

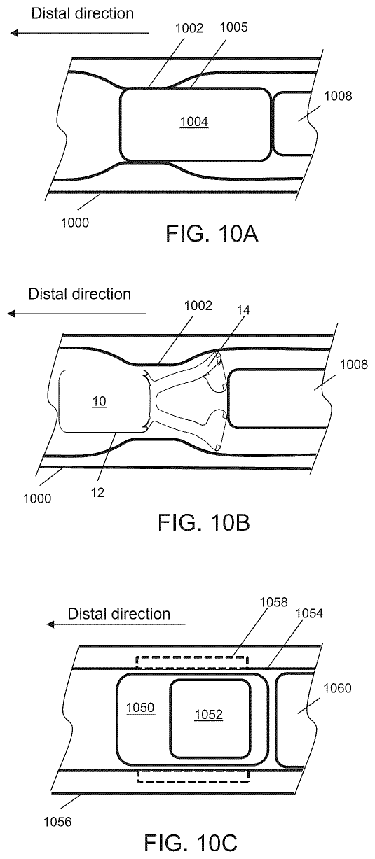

Examples described above include markers that have resilient prongs that extend through apertures in the cannula for retaining the marker in the cannula. Other examples retain the marker without requiring the apertures in the cannula. FIG. 10A illustrates an example in which a cannula 1000 includes a narrowed region 1002 that restricts distal movement of marker 1004. For example, the narrowed region 1002 may be slightly narrower than the diameter of at least a portion of a body 1005 of the marker 1004 such that the body 1005 engages with the narrowed region 1002, providing a friction fit between the narrowed region 1002 and the body 1005. A distal force applied by an obturator 1008 may overcome the frictional force between the narrowed region 1002 and the marker 1004 such that the marker can move distally past the narrowed region 1002 and deploy from the cannula 1000. Optionally, a marker with resilient prongs, such as marker 10 of FIG. 1A can be retained within cannula 1000 via engagement between the narrowed region 1002 and the resilient prongs 14. FIG. 10B illustrates an example in which a marker 10 is retained within cannula 1000. The body 12 can be narrower in diameter than the narrowed region 1002 such that only the resilient prongs 14 retain the marker 10 within the cannula 1000. The resilient prongs 14, when positioned within cannula 1000 proximally of the narrowed region 1002, are larger in diameter than the narrowed region 1002 such that engagement between the resilient prongs 14 and the narrowed region 1002 retains the marker 10 within the cannula 1000. Upon distal force applied by the obturator 1008, the resilient prongs 14 may be compressed by the narrowed region 1002, enabling the marker 10 to slide past the narrowed region 1002. In some examples, the resilient prongs 14 are in a compressed state when positioned in the cannula 1000 proximally of the narrowed region 1002 and are further compressed by the narrowed region 1002 when pushed past the narrowed region. Once deployed, the resilient prongs 14 uncompress to a deployed state in which the resilient prongs 14 are spread further apart than when positioned in the cannula 1000 proximally of the narrowed region 1002. In other examples, the resilient prongs 14 are in an uncompressed state when positioned in the cannula 1000 proximally of the narrowed region 1002.

The narrowed region 1002 can be formed in any suitable manner. In some examples, the narrowed region 1002 is formed by indenting the wall of the cannula 1000 inwardly. For example, the wall of the cannula 1000 can be punched inwardly in two diametrically opposite locations. In other examples, the narrowed region 1002 is formed by inserting an insert into the lumen of the cannula 1000. For example, an insert can be press fit into the lumen of the cannula 1000.

FIG. 10C illustrates an example in which a marker 1050 includes one or more magnets 1052, such as discussed above with respect to FIG. 10, that are configured to magnetically attach to the inner surface 1054 of a cannula 1056 to retain the marker 1050 within the cannula 1056. The cannula 1056 may include one or more ferromagnetic regions 1058 to which the magnets 1052 magnetically attach. Distal force applied to the marker 1050 by an obturator 1060 can overcome the magnetic force between the magnets 1052 and the ferromagnetic regions 1058 and deploy the marker 1050 from the cannula 1056.

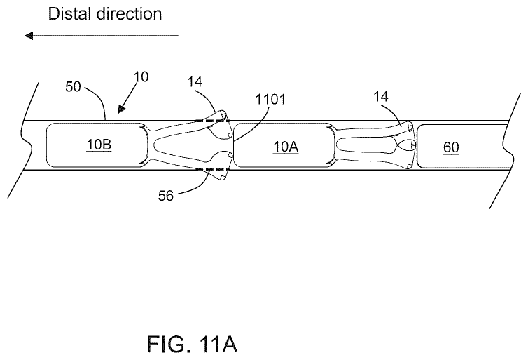

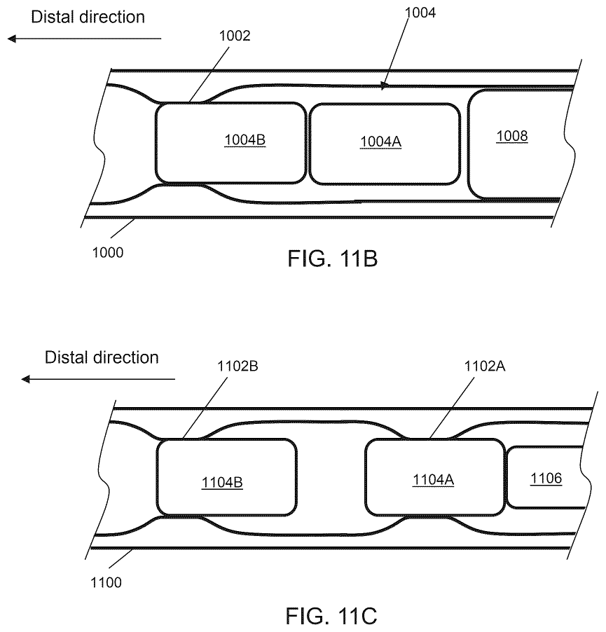

Optionally, a cannula, such as any of the cannulas described above can be loaded with multiple markers, including any of the markers discussed above. By loading a cannula with multiple markers, a surgeon can place multiple markers (e.g., markers at different locations) in a subject using a single cannula. An example of multiple markers loaded into a cannula is illustrated in FIG. 11A in which cannula 50 is loaded with multiple markers 10, including a proximal marker 10A and a distal marker 10B. Distal marker 10B is retained via engagement between resilient prongs 14 and apertures 56. Proximal marker 10A is located proximally behind distal marker 10B, with its resilient prongs 14 in a compressed state. Obturator 60 can be moved distally to apply a distal force to proximal marker 10A, causing proximal marker 10A to apply a distal force to distal marker 10B. The distal end 1101 of the markers 10 may be configured to engage with the resilient prongs 14 of adjacent markers 10 in similar fashion to the obturator 60 such that the proximal marker 10A can apply a force to the resilient prongs 14 of the distal marker 10B to cause the distal marker 10B to move distally and the resilient prongs 14 to compress. Continued distal movement of the obturator 60 can push the markers 10A and 10B distally and eject distal marker 10B from the cannula 50. The resilient prongs 14 of the proximal marker 10A may expand into the apertures 56, retaining the proximal marker 10A within the cannula 50. The cannula 50 can then be repositioned within the subject such that the proximal marker 10A can be placed in a different position within the subject than the distal marker 10B. In some examples, the cannula 50 includes multiple sets of apertures 56 spaced in a longitudinal direction of the cannula 50 such that each of multiple markers 10 is retained by its own set of apertures.

As noted above, any of the cannulas described herein can be loaded with multiple markers. FIG. 11B illustrates cannula 1000 of FIG. 10A loaded with multiple markers 1004. A proximal marker 1004A is positioned proximally of a distal marker 1004B. Distal movement of the obturator 1008 applies a distal force to a proximal marker 1004A, causing the proximal marker 1004A to apply a distal force to distal marker 1004B. With sufficient distal force, distal marker 1004B will be pushed past the narrowed region 1002 and out of the cannula 1000, deploying distal marker 1004B. The proximal marker 1004A will then be retained by narrowed region 1002. Further distal movement of the obturator 1008 can be used to deploy marker 1004A.

Optionally, a cannula has multiple retention features for retaining multiple markers within. FIG. 11C illustrates an exemplary cannula 1100 that is similar to cannula 1000 of FIG. 10A but includes multiple narrowed regions-narrowed region 1102A and narrowed region 1102B—for retaining multiple markers within the cannula 1100. Proximal marker 1104A is retained by narrowed region 1102A and distal marker 1104B is retained by narrowed region 1102B. Obturator 1106 can be used to apply a distal force to proximal marker 1104A, which will push proximal marker 1104A past narrowed region 1102A. As proximal marker 1104A is pushed distally by the obturator 1106, proximal marker 1104A will push against distal marker 1104B, pushing distal marker 1104B distally. Distal marker 1104B can be pushed past narrowed region 1102B and deployed from the cannula 1100. Proximal marker 1104A will then be retained by narrowed region 1102B. The cannula 1100 can then be repositioned within a subject to place the proximal marker 1104A at a different location within the subject. The proximal marker 1104A will be retained by the narrowed region 1102B during repositioning of the cannula 1100. The obturator 1106 can then be used to push the proximal marker 1104A past the narrowed region 1102B, deploying the proximal marker 1104A from the cannula 1100 and into the subject.

Although FIGS. 11A-C show two markers loaded within the respective cannulas, the cannulas and markers can be configured such that any desired number of markers can be loaded into the cannulas, including at least three, at least four, at least five, etc.

Any of the cannulas described herein can be used to place one or more of any of the markers described herein at one or more locations in tissue of a patient. A method for deployment of a marker may include inserting a cannula loaded with at least one marker into tissue of a subject at a desired location. The cannula can be any of the cannulas described herein, and the marker can be any of the markers described herein. The marker may be retained within the lumen of the cannula using any of the approaches described herein. For example, the marker may have resilient prongs that extend through apertures in the lumen to retain the marker in the lumen. Alternatively, the cannula may have a narrowed region that restricts distal movement of the marker, or the marker may have one or more magnets that magnetically attach to one or more regions of the cannula to retain the marker within the cannula. The method for deployment of the marker may include pushing an obturator distally within a lumen of the cannula to eject the marker from the cannula. For example, the obturator may apply a distal force to the resilient prongs of the marker to cause the resilient prongs to be compressed via the distal edges of the apertures of the cannula (e.g., angled distal edge 70). The resilient prongs may compress to their compressed state and the marker may be ejected distally out of a distal end of the cannula. In some examples in which multiple markers are loaded into a cannula, the obturator may push a proximal marker, which may push an adjacent marker. A distal-most marker may be ejected from the cannula and an adjacent marker may take the ejected marker's position within the cannula (e.g., retained by engagement between its resilient prongs and the apertures of the cannula, retained by the narrowed region, or retained via magnetic engagement with the cannula). Optionally, a cannula loaded with multiple markers can be repositioned to one or more additional locations of the subject and one or more additional markers can be deployed at the one or more additional locations by pushing the obturator further distally within the cannula.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A deployment system for a magnetic marker, the system comprising:
a cannula having a lumen, an open distal end, and a at least one lateral aperture; and
a magnetic marker having a body and at least one resilient prong extending from the body, the at least one resilient prong configured to have an expanded state and a compressed state, wherein the magnetic marker is configured to be stored in the expanded state within the lumen of the cannula such that at least a portion of the at least one resilient prong is within a corresponding lateral aperture of the at least one lateral aperture of the cannula,
wherein the lumen has a diameter configured to compress the at least one resilient prong of the magnetic marker into the compressed state during deployment of the magnetic marker.

2. The deployment system of claim 1, wherein the magnetic marker has two resilient prongs, and the cannula has two lateral apertures.

3. The deployment system of claim 1, wherein the body of the magnetic marker comprises a permanent magnet.

4. The deployment system of claim 3, wherein the permanent magnet has a polymer overmold.

5. The deployment system of claim 1, further comprising an obturator for insertion into a proximal end of the lumen and configured to push the magnetic marker during deployment.

6. The deployment system of claim 1, wherein the at least one resilient prong of the magnetic marker has an extension.

7. The deployment system of claim 6, wherein the extension comprises a projection that is configured to contact an inner surface of the lumen when the at least one resilient prong is in the compressed state within the lumen.

8. The deployment system of claim 1, wherein the expanded state is the same as a deployed state of the marker.

9. The deployment system of claim 1, wherein the at least one lateral aperture comprises a distal edge that is oriented at an angle that is non-perpendicular with a longitudinal direction of the cannula.

10. The deployment system of claim 1, wherein the magnetic marker comprises a drug.

11. The deployment system of claim 1, comprising a plurality of magnetic markers stored within the lumen.

12. A marker comprising:
a body; and
at least one resilient prong extending from the body, the at least one resilient prong configured to have an expanded state and a compressed state, wherein the marker is configured to be stored in the expanded state within a lumen of a cannula such that at least a portion of the at least one resilient prong is within a corresponding lateral aperture of a at least one lateral aperture of the cannula.

13. The marker of claim 12, wherein the marker has two resilient prongs.

14. The marker of claim 12, comprising a permanent magnet.

15. The marker of claim 14, wherein the permanent magnet has a polymer overmold.

16. The marker of claim 12, wherein the at least one resilient prong has an extension.

17. The marker of claim 16, wherein the extension comprises a projection that is configured to contact an inner surface of the lumen when the at least one resilient prong is in the compressed state within the lumen.

18. The marker of claim 12, wherein the marker comprises a drug.

19. A method for deployment system of a marker comprising:
positioning a cannula at a location of a subject, the cannula having stored therein a marker, the at least one marker having a body and at least one resilient prong extending from the body, the at least one resilient prong configured to have an expanded state and a compressed state, wherein the magnetic marker is stored in the expanded state within a lumen of the cannula such that at least a portion of the at least one resilient prong is within a corresponding lateral aperture of at least one lateral aperture of the cannula; and
moving an obturator distally to eject the marker from the cannula and deploy the marker at the location of the subject.

20. The method of claim 19, wherein the magnetic marker has two resilient prongs, and the cannula has two lateral apertures.
